(19) United States
(12) Patent Application Publication
(10) Pub. No.: US 2016/0041156 A1
(43) Pub. Date: Feb. 11, 2016

(54) Integrated Device for Measuring Multiple Analytes

(71) Applicant: OncoGenesis Inc., Morgan Hill, CA (US)

(72) Inventors: Christopher E. Todd, Campbell, CA (US); Nam Woo Kim, San Jose, CA (US); Peter Paul Gombrich, Salinas, CA (US)

(21) Appl. No.: 14/823,981

(22) Filed: Aug. 11, 2015

Related U.S. Application Data
(60) Provisional application No. 62/036,040, filed on Aug. 11, 2014.

(51) Int. Cl. G01N 33/543; G01N 21/25; G01N 27/416; G01N 21/64

(52) U.S. Cl. CPC G01N 33/54326; G01N 21/6428; G01N 21/25; G01N 27/416

(57) ABSTRACT

Integrated Multiplexed Point-of-Care device for measuring levels of target analytes from biological samples for determination of disease state. Specifically, a platform with a self contained fluidic cartridge comprising direct sample introduction, onboard sample processing, and multiplexed analyte detection is described.

### CROSS REFERENCE TO RELATED APPLICATIONS

This application claims the benefit of, and priority to, U.S. Provisional Patent Application Ser. No. 62/036,040, filed Aug. 11, 2014, titled “INTEGRATED DEVICE FOR MEASURING MULTIPLE ANALYTES,” the entire disclosure of which is hereby incorporated by reference in its entirety.

### FIELD OF THE INVENTION

This disclosure relates generally to an integrated device for processing raw biological materials and detecting target proteins and/or nucleic acids from biological samples for markers associated with screening and/or diagnosis of disease states. More specifically, embodiments of a device comprising a self-contained cartridge that allows for both sample processing and multiple analyte detection based on an electrochemical assay are described.

### BACKGROUND

Commonly used technologies currently in use for detecting target proteins and/or biological samples rely on either antigen or DNA/RNA analysis based primarily on optical detection such as fluorescence or chemiluminescence. In addition, these techniques classically require some type of sample preparation prior to introduction into the testing device.

Such commonly used technologies have a number of limitations, and new techniques and developments are a continued need.

### SUMMARY

Embodiments of the present invention provide a platform for sample processing and multiple analyte detection based on an electrochemical assay. In some embodiments, a device is provided comprising a self-contained cartridge that allows for both sample processing and multiple analyte detection based on an electrochemical assay.

In one embodiment of the platform, the device provides onboard sample preparation prior to downstream detection and/or quantification of analytes. Processing can include sample isolation, cell concentration and cell lysis all within the device.

In another embodiment, the device can process a sample for detection with c volumes as small as 1 μl per analyte.

In yet another embodiment, the device isolates and detects multiple analytes of interest (biomarkers) simultaneously.

In another aspect, embodiments utilize multiplexed electrochemical detection to determine analyte presence and/or concentration. Signal levels are measured in the range of picoamps.

### BRIEF DESCRIPTION OF THE DRAWINGS

Other aspects, embodiments and advantages of the invention will become apparent upon reading of the detailed description of the invention and the appended claims provided below, and upon reference to the drawings in which:

FIG. 1 is a flow chart of sample preparation within the device, according to some embodiments of the present invention.

FIG. 2 is a schematic side view showing processing of large sample volumes, collecting the sample cells and transitioning small antigen volumes into downstream sample processing, according to some embodiments of the present invention.

FIG. 3 illustrates the sample separated into multiple analyte detection channels, according to some embodiments of the present invention.

FIG. 4 is a prospective view which depicts multiplexed electrochemical sensors with mating interface to fluidic device, according to some embodiments of the present invention.

FIGS. 5A and 5B depict top and cross-sectional views, respectively, of the internal design of the electrochemical sensor, according to some embodiments of the present invention.

FIG. 6 is a schematic drawing illustrating redox amplification to transfer a biological chemical reaction to an electrical current.

### DETAILED DESCRIPTION

Embodiments of the present disclosure provide an integrated, disposable cartridge device for processing patient samples to identify and/or quantify multiple analytes to provide diagnostic and eventually therapeutic intervention for different disease states. Of particular advantage, the device is a point-of-care (POC) device. The device provides a self-contained system to introduce the sample and sample preparation for downstream analyte detection. The device allows direct sample introduction and eventual processing of small antigen volumes to reduce processing time (incubation rates) and reagent costs. The device homogeneously separates the sample into multiplexed reaction wells to determine biomarker presence and/or concentration. Finally, the multiplexed electrochemical detection sensors eliminate the need for complicated optical detection systems, thus reducing the often difficult analyte amplification methods required to detect small analyte concentrations with optical-based systems.

In some embodiments, a cartridge device is provided. The cartridge device may be comprised of and input port for injecting unprocessed samples into the cartridge, a processing chamber that concentrates biological materials using a filter, a reaction chamber where the processed samples are reacted with detection reagents; and detection chamber where the target analyte is detected.

In some embodiments, the number of reaction chamber and detection chambers are equal to the number of analytes being tested.

The process chamber may be configured to include means to lyse outer membranes for cells from the biological materials. Cells may be lysed by any suitable manner, for example the cells are lysed with detergents. Alternatively, the cells are lysed with mechanical shearing.

In some embodiments, the cartridge includes a fluidic circuit to flow materials from one chamber to the next. The fluidic circuit may be configures to provide a flow volume of the fluidic circuits of 1 μl per analyte and larger.

In one embodiment, the reaction chamber further includes reagents that are dried onto the chamber and reconstitutes to an active state with introduction of liquid solution. In this configuration, the reagents may be capture and detector antibodies for the target analytes. In an exemplary embodiment, the reagents are oligonucleotide primers.

In some embodiments, one antibody or oligonucleotide is immobilized onto a magnetic bead. When the analyte is captured onto the magnetic beads, it can be manipulated inside the fluidic circuits by magnets.

In some embodiments, the one or more detection chambers include a sensor that detects the level of analytes in the reaction chamber. The sensor may be configured to detect absorbance, fluorescence, luminescence or refraction of light.

Any suitable sensor may be used, such as for example an electrochemical sensor, or an optical sensor. When an electrochemical sensor is used it may be comprised of a carbon 3D Inter-Digitated Electrode Array (3D IDEA).

In another aspect, a platform, system or instrument is provided which is comprised of an interface for receiving the cartridge above. Generally, the instrument further includes electrical, magnetic, optical, and mechanical inputs to the cartridge; a circuit for measuring the signal from the sensor; a program to execute instructions to the cartridge; and data output from the sensors. In some embodiment, the data output is wireless.

### Sample Preparation and Antigen Separation:

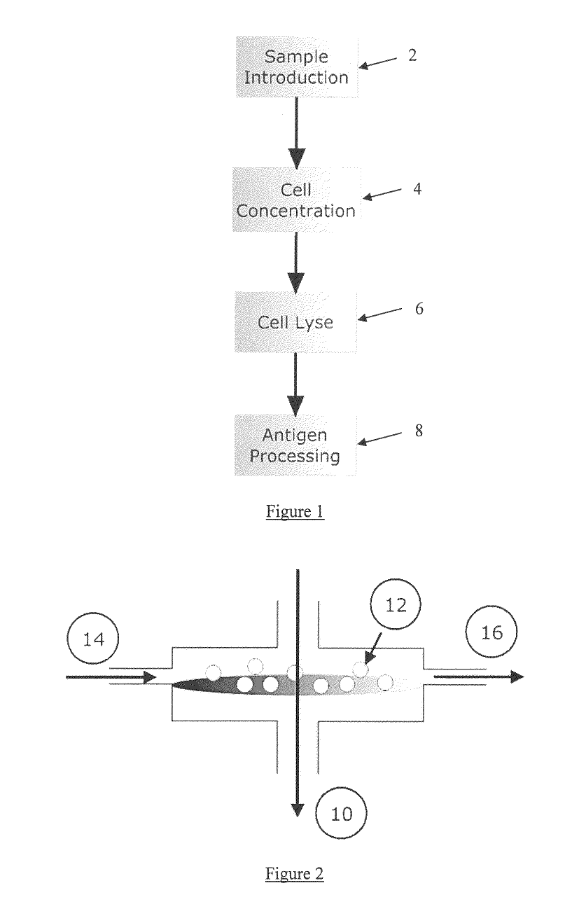

Part of the embodiment of the present disclosure employs sample introduction that is simple enough to allow processing by minimally trained personnel. A preferred embodiment uses a simple sample introduction device such as a bulb pipette. Bulb pipettes are easy for untrained personnel to extract desired sample volumes from collection devices such as a vial. Referring to FIG. 1, the point-of-care (POC) fluidic cartridge may include a sample introduction opening where the technician introduces the sample directly into the cartridge at step 2. With the fluidic cartridge inserted into the instrument, sample preparation occurs by separating desired cells or analytes from the collection fluids. An embodiment of sample separation can be achieved by membrane filter to collect the desired material and flowing other material into a contained waste well. In this embodiment, the filter membrane acts as a cell concentrator at step 4. Appropriate filter pore sizes may be optimized to isolate analytes other than cells. If cells are the desired sample, the filter membrane can act as a supporting media for cell lyses at step 6, and transfer into the small volume processing section of the fluidic device at step 8.

FIG. 2 shows a schematic representation with the large volume sample introduction flowing through a membrane to concentrate cells 12 and remove other material into waste collection 10. Onboard lyse solution introduced to membrane containing cells for incubation, releasing internal analytes 14. After cell lyses, released analytes are flushed into the low volume section of the fluidic cartridge for further processing 16.

### Multiplexed Analytes Capture:

To measure multiple analytes with ELISA type assays, a separate reaction well is utilized containing immobilized capture antibodies specific for the desired biomarker. FIG. 3 illustrates the flow of sample into the reaction wells. To increase sensitivity, incubation is required for analyte capture. To decrease incubation times, and therefore overall processing time, selective reaction well geometry is used to maximize analyte capture by immobilized antibodies. One embodiment minimizes bulk sample volumes by increasing capture antibody surface area of the reaction well. With reduced bulk sample volumes, analyte capture by diffusion is maximized. Another embodiment utilizes a flow through sample introduction method. In this configuration, the analyte is flowed across the capture antibodies for maximum capture efficiency instead of capture by diffusion. Reaction well design constitutes a long narrow channel with capture antibodies throughout. Incubation occurs under sample flow conditions instead of static flow.

### Electrochemical Biomarker Detection:

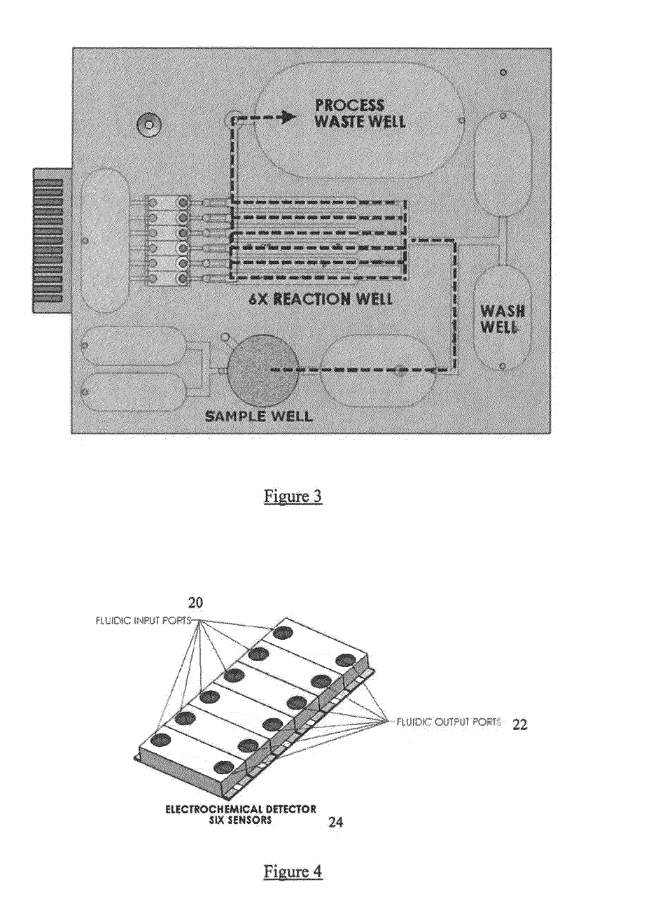

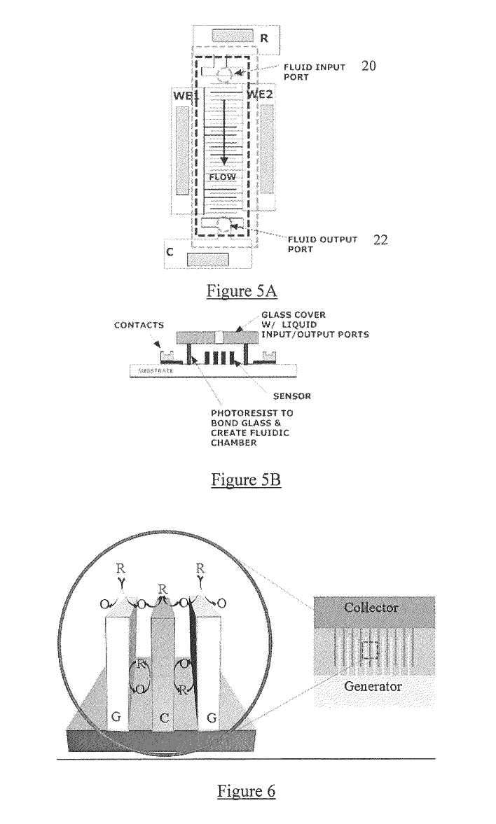

Biomarker presence or quantification utilizes an electrochemical sensor device. One sensor is fluidically mated to each biomarker reaction well. FIG. 4 shows a multiplexed series of sensors and assembled into a fluidic package, utilizing multiple fluidic input ports 20, multiple fluidic output ports 22 and multiple detection sensors 24. After sample incubation, a representative amount of sample is flowed across the sensor creating an electrical signal proportional to biomarker concentration. The preferred embodiment uses a three dimensional InterDigitated Electrode Array (3D IDEA) and Redox Amplification. 3D IDEA sensor is a carbon sensor optimized to electrically measure substrate (biomarker) flowed across the interdigitated electrodes. A large electrical measuring surface area allows amplified signal measuring as compared to planar IDEA devices. FIGS. 5A and 5B show top and cross-sectional schematic views, respectively, of an IDEA sensor assembled into a fluidic chamber. Additionally, redox amplification is a well known technique to transfer a biological chemical reaction to an electrical current as illustrated in FIG. 6. A major advantage of the IDEA electrochemical biosensor over the typical optical detection method is that the electrochemical biosensor offers simpler measurements that can operate in turbid solutions that provide significant advantages in the analysis of biological samples. The signals from electrochemical biosensors can be detected in IDEA sensor by redox amplification, and since the amplified signal is purely electrical in nature, the potential background noise from biological samples is significantly reduced. Furthermore, the IDEA sensor can be miniaturized to fit any detection format. This mode of detection provides the improved stability and robust functionality demanded of point of care systems while providing high signal to noise ratio.

In this system, as in typical ELISA, the antigen captured by an antibody and detected by another antibody labeled with alkaline phosphatase (ALP) can be detected by IDEA using a substrate, such as p-aminophenylphosphate (p-APP), whose end product is electroactive [p-aminophenol (p-AP)]. The electroactive end product (p-AP) is then detected and the signal is amplified by IDEA sensor. When electroactive product is flowed over the IDEA sensor, a reversible reaction occurs within the electrode creating an electrical current between the electrodes. The reaction regenerates when dispersed into the bulk solution and back to the electrodes. Since the signal amplification is electronic, it has vastly reduced noise from the biological matrix resulting in high sensitivity with low signal-to-noise. Signals from each biomarker or control will be detected by an individual biosensor inserted into each detection chamber. Due to the simplicity and manufacturability of the IDEA biosensor, the cost of the biosensors will be minimal. By having the disposable biosensors embedded into the test cartridge, the complexity, size, and the cost of the instrument to run the test cartridge is greatly reduced. Initial studies of the sensor shows greater than 100-fold sensitivity in the detection limit of the 3D carbon IDEA sensor when compared to the standard fluorescent or absorbance detection method indicating significant promise in detecting low-level protein targets in complex biological materials.

The present invention is not to be limited in scope by the specific embodiments and examples disclosed herein which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

What is claimed is:

1. An integrated device for processing complex samples and detecting multiple analytes for indication of disease state.

2. A cartridge device, the cartridge comprised of:
one or more input port for injecting unprocessed samples into the cartridge;
one or more processing chambers that concentrates biological materials using a filter;
one or more reaction chambers where the processed samples are reacted with detection reagents; and
one or more detection chambers where the target analyte is detected.

3. The device of claim 2 wherein the number of reaction chambers and detection chambers are equal to the number of analytes being tested.

4. The device of claim 2 wherein the processing chamber includes means to lyse outer membranes for cells from the biological materials.

5. The device of claim 4 where the cells are lysed with detergents.

6. The device of claim 4 where the cells are lysed with mechanical shearing.

7. The device of claim 2 wherein the cartridge includes fluidic circuit to flow materials from one chamber to the next.

8. The device of claim 7 wherein the flow volume of the fluidic circuits are 1 μl per analyte and larger.

9. The device of claim 2 wherein the reaction chamber includes reagents that are dried onto the chamber and reconstitutes to an active state with introduction of liquid solution.

10. The device of claim 9 wherein the reagents are capture and detector antibodies for the target analytes.

11. The device of claim 9 wherein the reagents are oligonucleotide primers.

12. The device of claim 9 wherein one antibody or oligonucleotide is immobilized onto a magnetic bead.

13. The device of claim 12 wherein the analyte captured onto the magnetic bead is manipulated inside the fluidic circuits by magnets.

14. The device of claim 2 wherein the detection chamber includes a sensor that detects the level of analytes in the reaction chamber.

15. The device of claim 14 wherein the sensor is electrochemical sensor.

16. The device of claim 15 wherein the electrochemical sensor is a carbon 3D Inter-Digitated Electrode Array (3D IDEA).

17. The device of claim 14 wherein the sensor is an optical sensor.

18. The device of claim 17 wherein the sensor detects absorbance, fluorescence, luminescence or refraction of light.

19. An instrument comprising:
an interface for receiving the cartridge described in claim 1;
electrical, magnetic, optical, and mechanical inputs to the cartridge;
a circuit for measuring the signal from the sensor;
program to execute instructions to the cartridge; and
data output from the sensors.

20. The instrument of claim 19 wherein the data output is wireless.
